(19) United States
(12) Patent Application Publication
(10) Pub. No.: US 2024/0182827 A1
(43) Pub. Date: Jun. 6, 2024

(54) CONTAINER AND SYSTEM FOR CULTURING PHOTOTROPHIC MICROORANISMS

(71) Applicant: ALGIECEL APS, Copenhagen (DK)

(72) Inventors: Henrik BUSCH-LARSEN, Odense M (DK); Niels-Henrik NORSKER, Vester Skerninge (DK); Robert Emil LARSEN, Virum (DK)

(21) Appl. No.: 18/556,181

(22) PCT Filed: Apr. 20, 2022
(86) PCT No.: PCT/EP2022/060426

§ 371 Date: Oct. 19, 2023

(30) Foreign Application Priority Data
Apr. 20, 2021 (DK) PA 2021 00394

(51) Int. Cl. C12M 1/00; C12M 1/12

(52) U.S. Cl. CPC C12M 21/02; C12M 23/06; C12M 23/22; C12M 23/52; C12M 31/02; C12M 31/10

(57) ABSTRACT

The present invention relates to a container comprising at least one gas-inlet, a photobioreactor and at least one light source, wherein the at least one gas-inlet of the container is in fluid connection with at least one gas-inlet of the photobioreactor and wherein the at least one light source is placed in a distance less than 5 cm from an outer surface of a photobioreactor tube and/or the at least one light source is placed inside the photobioreactor tube.

### TECHNICAL FIELD OF THE INVENTION

The present invention relates to a container or a system for culturing a phototrophic microorganism. In particular, the present invention relates to a container or a system comprising a photobioreactor for culturing a phototrophic microorganism.

### BACKGROUND OF THE INVENTION

For years alternatives to fossil fuels have been investigated, since fossil fuels are harmful to the environment and responsible for most of the CO₂ released into the atmosphere with significant consequences for the environment.

Therefore, researchers have been looking for alternative energy sources and ways to provide energy sources which may be CO₂-reducing or CO₂-neutral to the environment.

Cultivated microalgae, in the present algae, represent a promising source of such energy supplies.

Biomass derived from algae can be used to produce energy as a raw material for combustion or co-incineration with other waste fuels or through the production of biofuels.

Biofuels derived from algae can take the form of pyrolytic solid fuels, flammable gases (hydrogen and methane) or liquid hydrocarbons and biodiesel.

Since algae are photogenic microorganisms, they grow by consuming carbon dioxide (CO₂) and energy from light using the mechanism described as photosynthesis. Therefore, cultivating algae may also have desirable side effects on carbon sequestration and the environment.

Furthermore, algae are a diverse group of photogenic microorganisms that play an important role in the biosphere. They are characterized by a high growth rate potential compared to other typical energy crops. They are increasingly used in agriculture, environmental protection, medicine and energy production. Many systems have been described for the production of algae and the cultivation products derived from this production often have a low efficiency, low productivity and low yields due to suboptimal production process parameters, or the production facilities require large areas with difficulties in up- or down-scaling of the production capacity.

Parameters of the production process, such as nutrients supplied during production concentration of nutrients, pH, CO₂ and temperature may be important, high productivity of good-quality algae, optimal growth rate and high productivity of cultivation products may depend on factors like distribution and availability of light with the appropriate wavelength and intensity.

Algae are a potential source of renewable energy and a raw material for the production of biofuels, but unfortunately, the production method presently available are very energy-intensive making cultivation products like biofuel production uneconomical, with low productivity and has a large footprint and with difficulties in up- or down-scaling of the production capacity.

Thus, there is a need in the industry for improved systems and methods for fermenting photogenic microorganisms, like algae, to improve productivity and make the production of e.g. biodiesel more economically interesting.

Hence, an improved photobioreactor construction, an improved system, and an improved method for cultivating photogenic microorganisms, like algae (microalgae), and providing cultivation products, like biodiesel, would be advantageous. In particular, a more efficient, economic, photobioreactor construction, system, and method providing a better light distribution, more even light exposure of the photogenic microorganisms, higher quality and higher productivity and/or improved options for up- or down-scaling according to production capacity and/or at the same time limiting the footprint occupied by the photobioreactor or the system, would be advantageous.

### SUMMARY OF THE INVENTION

Thus, an object of the present invention relates to a compact design such as a container and a system comprising a photobioreactor for culturing a phototrophic microorganism.

In particular, it is an object of the present invention to provide a container and a system comprising a photobioreactor for culturing a phototrophic microorganism that solves the above-mentioned problems of the prior art with efficiency, economy, light exposure of the photogenic microorganisms, quality, productivity, options for up- or down-scaling according to production capacity and/or the large footprint occupied by the photobioreactor or the system.

Thus, one aspect of the invention relates to a container comprising at least one gas-inlet, a photobioreactor and at least one light source, wherein the at least one gas-inlet of the container is in fluid connection with at least one gas-inlet of the photobioreactor and wherein the at least one light source is placed in a distance less than 5 cm from an outer surface of a photobioreactor tube and/or the at least one light source is placed inside the photobioreactor tube.

Another aspect of the present invention relates to a system comprising two or more containers according to the present invention, wherein the two or more containers are in fluid contact via at least one cultivation medium-throughput and/or a cultivation broth throughput.

A further aspect of the present invention relates to a system comprising one or more containers according to anyone of claims 1-10, wherein the one or more containers are in fluid contact via at least one cultivation medium-throughput and/or via at least one cultivation broth-throughput, with:
one or more further containers according to anyone of claims 1-10;
one or more downstream processing container;
one or more pump containers; and/or
one or more containers comprising the cooling system.

Yet another aspect of the present invention relates to a method for producing at least one cultivation product from culturing one or more phototrophic microorganism, the method comprising the steps of:
(i) Providing the one or more phototrophic microorganism to a photobioreactor of the container according to the present invention or the system according to the present invention;
(ii) Allowing the one or more phototrophic microorganism to cultivate under time, temperature and illumination conditions suitable for providing a cultivation broth comprising the cultivation product; and
(iii) Isolating the at least one cultivation product from the cultivation broth.

### BRIEF DESCRIPTION OF THE FIGURES

FIGS. 1a and 1b shows a container (1) according to the present invention. The container (1) comprises multiple photobioreactor tube modules (2) making up the photobioreactor. In the present embodiment the container (1) comprises 25 photobioreactor tube modules (2). Each photobioreactor tube module (2) comprises a multiple number of photobioreactor tubes (3) which are not shown in FIG. 1. Two or more of the 25 photobioreactor tube modules (2) may be serially connected increasing the length of the photobioreactor. The photobioreactor tube modules (2) may comprise two or more photobioreactor tubes (not shown); such as 5 or more photobioreactor tubes; e.g. 10 or more photobioreactor tubes; such as 25 or more photobioreactor tubes; e.g. 50 or more photobioreactor tubes; such as 75 or more photobioreactor tubes; e.g. 100 or more photobioreactor tubes; such as 110 or more photobioreactor tubes; e.g. 115 or more photobioreactor tubes. The photobioreactor tube modules (2) may have a length in the range of 0.1-15 m, such as in the range of 4-13 m, e.g. in the range of 6-12 m, such as in the range of 9-11 m, e.g. about 10 m. The container (1) may be a 20 feet container or a 40 feet container, preferably a 40 feet container,

FIG. 2 shows a single photobioreactor tube module (2) according to the present invention. The photobioreactor tube module (2) comprises 114 photobioreactor tubes (3). Each of the photobioreactor tubes (3) are connected, by one end of one photobioreactor tube (3a) may be connected to one end of a second photobioreactor tube (3b), e.g. by using a manifold or a U-shaped tube. The interspace between the photobioreactor tubes may form a polygonic shaped space (4) between the surrounding photobioreactor tubes (3) where the sides are circular shaped or partly circular shaped. The polygonic shaped space (4) provided by the present embodiment and as demonstrated here in FIG. 2 may comprise four sides (4a, 4b, 4c, and 4d). The light source (not shown in FIG. 2) may be placed in this polygonic shaped space (4) or as one or more sheaths (not shown in FIG. 2) surrounding each of the photobioreactor tubes (3) and placing the at least one light source between the at least two sheaths and the photobioreactor tube (3) and facing the photobioreactor tubes (directing the light into the photobioreactor tube (3)). The surrounding frame (5) of the photobioreactor tube module (2) may be provided with a light source according to the present invention to illuminate the periphery of the photobioreactor tubes (3). Reference (A) relates to a subset of the photobioreactor tube module (2) and the photobioreactor tubes (3) shown in FIG. 3.

FIG. 3 shows the subset (A) of the photobioreactor tube module (2) and the photobioreactor tubes (3) marked in FIG. 2. The subset of a photobioreactor tube module (2) also shows the embodiment of the present invention where the light source (6) is inserted into the polygonic shaped space (4) provided by the present embodiment may comprise four sides (4a, 4b, 4c, and 4d). The distance between the outer surface of the photobioreactor tube and the light source (the gap) may be less than 4 cm, such as less than 3 cm, e.g. less than 2 cm, such as less than 1 cm, e.g. less than 0.1 cm, such as less than 0.05 cm, e.g. less than 0.01 cm, such as 0 cm. The distance between the outer surface of the photobioreactor tube and the light source (the gap) have shown to affect the intensity and the efficiency of the light source and has been found to be kept at small as possible,

FIG. 4 shows a single photobioreactor tube module (2) according to the present invention. The photobioreactor tube module (2) comprises 114 photobioreactor tubes (3). Each of the photobioreactor tubes (3) are connected, by one end of one photobioreactor tube (3a) may be connected to one end of a second photobioreactor tube (3b), e.g. by using a manifold or a U-shaped tube. The interspace between the photobioreactor tubes may form a polygonic shaped space (4) between the surrounding photobioreactor tubes (3) where the sides are circular shaped or partly circular shaped. The polygonic shaped space (4) provided by the present embodiment may comprise three sides (not shown in FIG. 4). The light source (not shown) may be placed in the polygonic shaped space (4) or as one or more sheaths (not shown in FIG. 4) surrounding each of the photobioreactor tubes (3) and placing the at least one light source between the at least two sheaths and the photobioreactor tube (3) and facing the photobioreactor tubes (directing the light into the photobioreactor tube (3)). The surrounding frame (5) of the photobioreactor tube module (2) may be provided with a light source according to the present invention to illuminate the periphery of the photobioreactor tubes (3). Reference (B) relates to a subset of the photobioreactor tube module (2) and the photobioreactor tubes (3) shown in FIG. 5.

FIG. 5 shows the subset (B) of the photobioreactor tube module (2) and the photobioreactor tubes (3) marked in FIG. 4. The subset of a photobioreactor tube module (2) also shows the embodiment of the present invention where the light source (6) is inserted into the polygonic shaped space (4) provided by the present embodiment may comprise three sides (4a, 4b, and 4c). The distance between the outer surface of the photobioreactor tube and the light source (the gap) may be less than 5 cm, such as less than 4 cm, e.g. less than 3 cm, such as less than 2 cm, e.g. less than 1 cm, such as less than 0.5 cm, e.g. less than 0.01 cm, such as 0 cm. The distance between the outer surface of the photobioreactor tube and the light source (the gap) have shown to affect the intensity and the efficiency of the light source and has been found to be kept at small as possible, and

FIG. 6 shows embodiment of including the light source (6) into the photobioreactor construction according to the present invention. The light source (6) may be placed in one or more sheaths (7) surrounding each of the photobioreactor tubes (3) and placing the at least one light source between the at least two sheaths and the photobioreactor tube (3) and facing the photobioreactor tubes (directing the light into the photobioreactor tube (3)). The embodiment shown in FIG. 4 comprises two sheaths (7) surrounding the photobioreactor tube (3) allowing the light source (6) to be facing the photobioreactor tube (3). The two sheaths (7) surrounding the photobioreactor tube (3) abuts each other in part (8) encircling the photobioreactor tube (3).

FIG. 7 shows a cross-sectional view of an embodiment of the present invention were a profile (9) may be placed in the space between the photobioreactor tubes (3). The profile (9) also comprises a hollow cavity (10) positioned and extending along the centre line C of the profile (9) which may be formed as an elongated element 2. In an embodiment of the present invention the hollow cavity (10) may extend in the full length of the profile (9), and preferably in the full length of the photobioreactor tube (3). The light source (6) may be attached on the surface of the profile (9) in a distance from the photobioreactor tube (3). The distance of the light source (6) from the outer surface of the photobioreactor tube (3) may be controlled by the arms (11) or from the distance provided from the collection of the photobioreactor tubes (3). The distance between the light source (6) and the outer surface of the photobioreactor tube (3) in FIG. 7 may be about 1 cm.

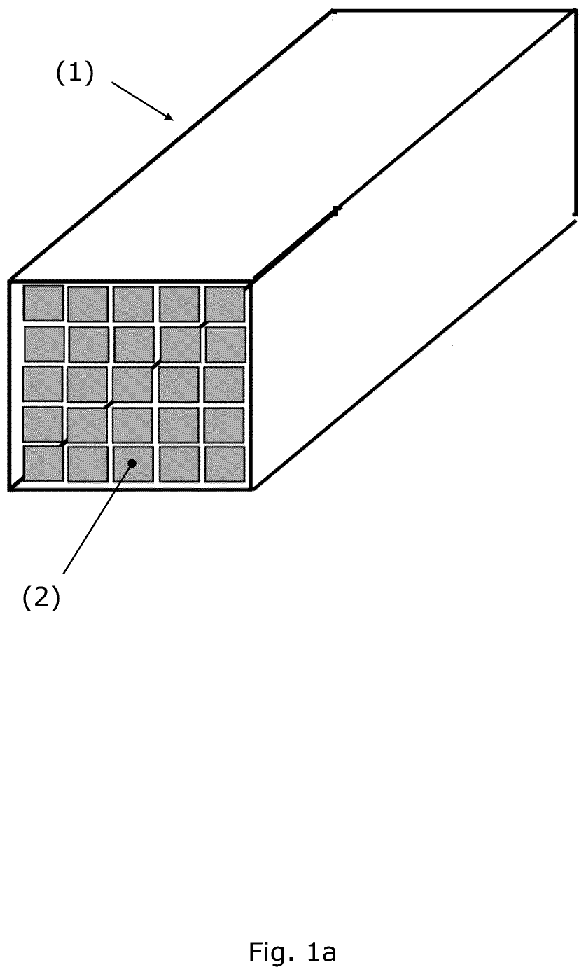

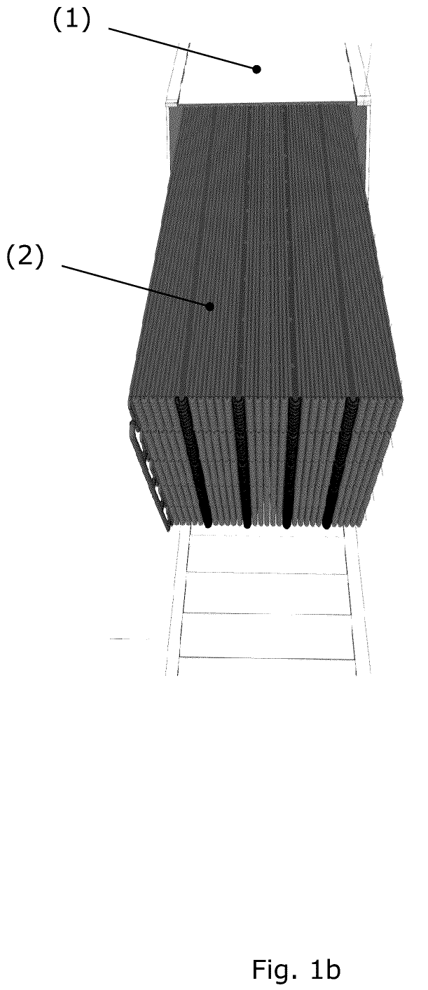

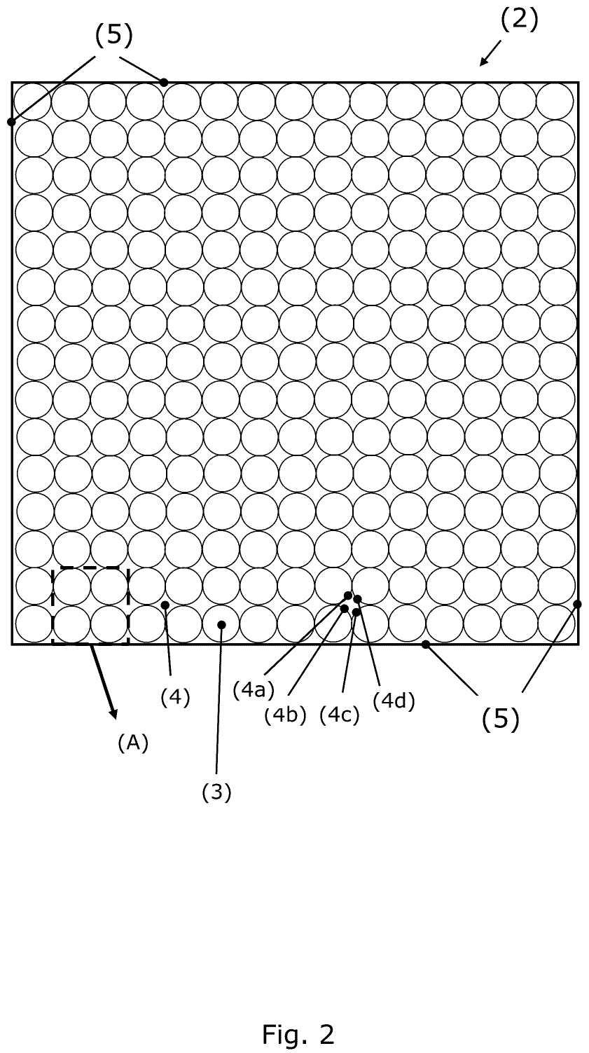

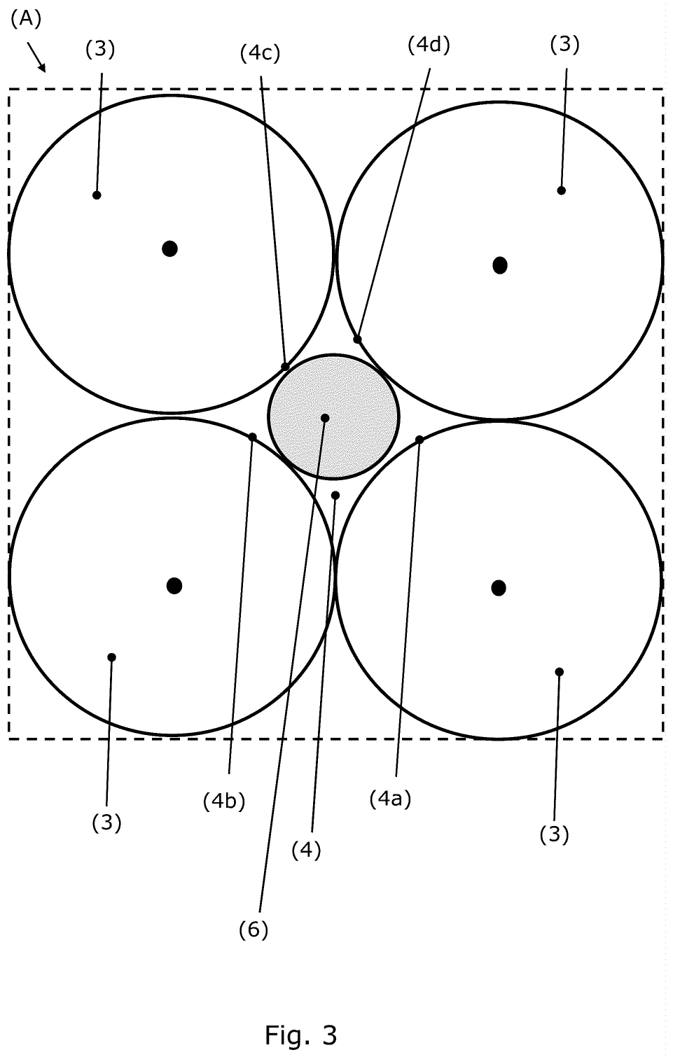

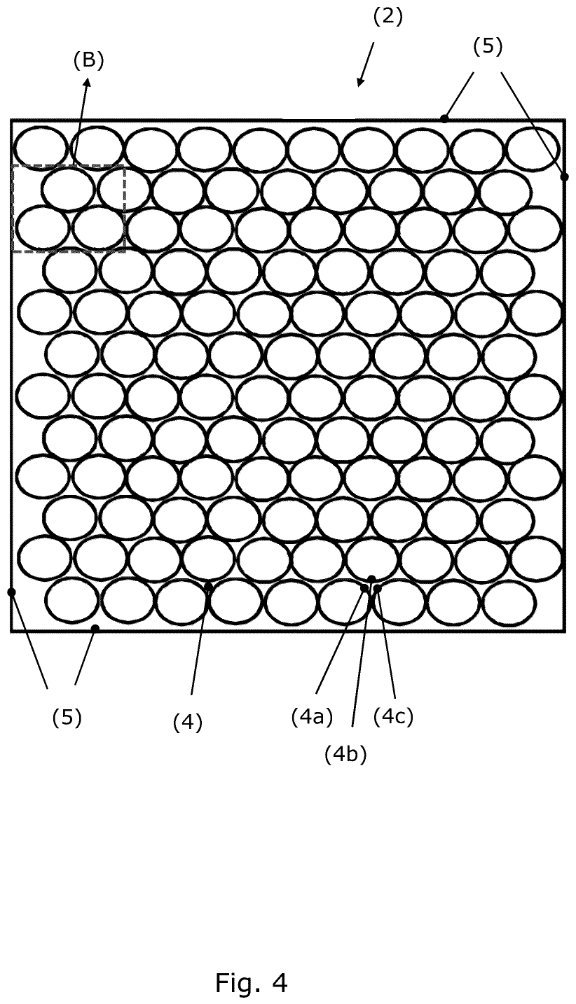

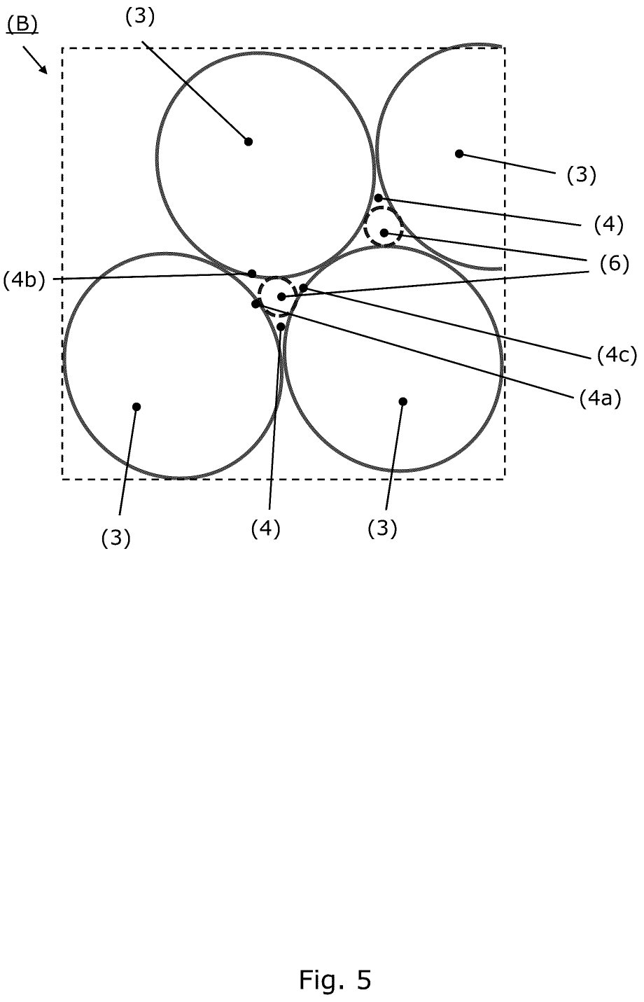

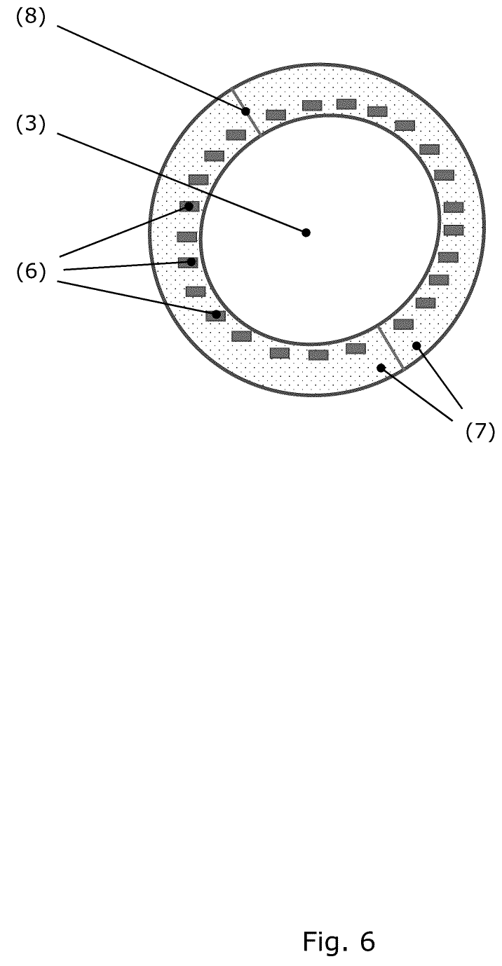

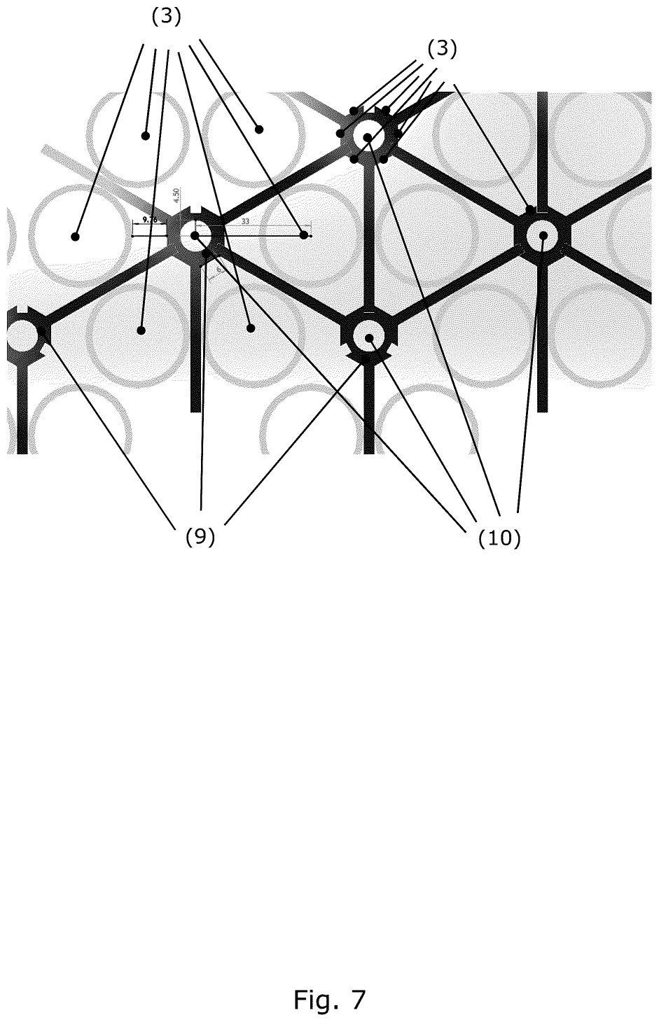

The present invention will now be described in more detail in the following.

### DETAILED DESCRIPTION OF THE INVENTION

Accordingly, the inventors of the present invention surprisingly found a new way to construct a photobioreactor which makes it possible to provide a highly flexible system for cultivating one or more phototrophic microorganisms in a method having a high productivity and a high effectivity in light source utilization.

In a preferred embodiment of the present invention relates to a container comprising at least one gas-inlet, a photobioreactor and at least one light source, wherein the at least one gas-inlet of the container is in fluid connection with at least one gas-inlet of the photobioreactor and wherein the at least one light source is placed in a distance less than 5 cm from an outer surface of a photobioreactor tube and/or the at least one light source is placed inside the photobioreactor tube.

Preferably, the light source may be placed outside the photobioreactor tube.

In an embodiment of the present invention the container may comprise a cultivation substrate inlet, a cultivation medium inlet, and/or a cultivation broth inlet.

When the container comprises a cultivation substrate inlet, the container may be in fluid connection with a container comprising a cultivation substrate. Cultivation substrate may be supplied to the photobioreactor when products are harvested from the photobioreactor or when the liquid level are reduced below a certain limit.

When the container comprises a cultivation medium inlet, the container may be in fluid connection with a downstream processing system (preferably the outlet of the downstream processing system) placed inside the container comprising the photobioreactor or in a separate downstream processing container. Cultivation medium may be supplied to the photobioreactor from the separator where the biomass has been removed, or substantially removed.

When the container comprises a cultivation broth inlet, the container may be in fluid connection with an outlet of another container. This allows upscaling of the system to include two or more containers comprising photobioreactors, light sources and gas inlets, which are in fluid connection.

In an embodiment of the present invention the container may comprise a cultivation broth outlet.

When the container comprises a cultivation broth outlet, the container may be in fluid connection with an inlet of another container. This allows upscaling of the system to include two or more containers comprising photobioreactors, light sources and gas inlets, which are in fluid connection.

In an embodiment of the present invention the system may comprise at least one container comprising the photobioreactor according to the present invention and one downstream processing container, such as at least two containers comprising the photobioreactor and one downstream processing container, e.g. at least 3 containers comprising the photobioreactor and one downstream processing container, such as at least 4 containers comprising the photobioreactor and one downstream processing container, e.g. at least 5 containers comprising the photobioreactor and one downstream processing container.

Preferably, the container may comprise a cooling water inlet.

In an embodiment of the present invention the cooling liquid may be cooling water. Preferably, the cooling liquid may be provided from a container comprising a cooling system or the cooling liquid, such as cooling water may be provided from a cooling tower in proximity to the system comprising at least one container comprising the photobioreactor.

In a further embodiment of the present invention the container may further comprise at least one gas-outlet.

Carbon dioxide (CO₂) may be supplied to the container comprising the photobioreactor and to the photobioreactor as such, via the gas-inlet.

Oxygen (O₂) may be obtained from the container comprising the photobioreactor and from the photobioreactor via the gas-outlet.

In an embodiment of the present invention the at least one gas-outlet of the container may be in fluid connection with at least one gas-outlet of the photobioreactor.

The photobioreactor may preferably comprise a transparent photobioreactor. Preferably, the photobioreactor tube according to the present invention may preferably comprise a transparent photobioreactor tube.

The transparent photobioreactor/transparent photobioreactor tube may allow light to pass through so that objects behind can be distinctly seen.

In an embodiment of the present invention the transparent photobioreactor/transparent photobioreactor tube may be a translucent photobioreactor or a translucent photobioreactor tube.

The term “translucent” may relate to a material allowing light to pass through but potentially diffusing the light so that objects on the other side cannot be distinguished.

The at least one light source may comprise an artificial light source. Preferably the artificial light source comprises LEDs. However, other artificial light sources are also suitable, for example fluorescent lamps, neon lamps, metal vapor lamps, inert gas lamps, halogen lamps, sulphur plasma lamps and light guide conducted natural daylight, e.g. using optical fibres.

During cultivation in the photobioreactor wavelengths and/or intensity of the means of lighting elements may be optimized in facilitate the desired cultivation.

Preferably, the photobioreactor and/or the photobioreactor tubes may be transparent when the at least one light source may be placed outside the photobioreactor, e.g. at a distance less than 5 cm from an outer surface of the photobioreactor; or when the at least one light source may be placed outside the photobioreactor tube and inside the photobioreactor tube.

The photobioreactor may preferably be provided with one or more photobioreactor tube modules; such as 2 or more photobioreactor tube modules; e.g. 4 or more photobioreactor tube modules; such as 5 or more photobioreactor tube modules; e.g. 10 or more photobioreactor tube modules; such as 15 or more photobioreactor tube modules; e.g. 20 or more photobioreactor tube modules; such as 25 or more photobioreactor tube modules.

One of the advantages of constructing the photobioreactor in multiple photobioreactor tube modules, e.g. two or more photobioreactor tube modules, is that inspection, replacement and/or repairment of smaller parts of the photobioreactor or individual photobioreactor tubes.

The use of photobioreactor tube modules allows for specific up- and down-scaling of the photobioreactor according to the need and depending on the product to be produced.

The one or more photobioreactor tube modules may comprise two or more photobioreactor tubes; such as 5 or more photobioreactor tubes; e.g. 10 or more photobioreactor tubes; such as 25 or more photobioreactor tubes; e.g. 50 or more photobioreactor tubes; such as 75 or more photobioreactor tubes; e.g. 100 or more photobioreactor tubes; such as 110 or more photobioreactor tubes; e.g. 115 or more photobioreactor tubes.

In an embodiment of the present invention one end of one photobioreactor tubes may be connected to one end of a second photobioreactor tube, preferably using a U-shaped tube or a manifold.

In yet another embodiment of the present invention the photobioreactor comprises a repeating number of photobioreactor tube modules continuously connected and/or a repeating number of photobioreactor tubes continuously connected. A set of repeating number of photobioreactor tubes continuously connected may form a photobioreactor tube module.

When the at least one light source is placed only inside the photobioreactor tube, the photobioreactor tube may or may not be transparent. In an embodiment of the present invention the at least one light source may be placed only inside the photobioreactor tube and the photobioreactor tube may comprise a light reflecting material, at least on the inside surface of the photobioreactor.

In an embodiment of the present invention wherein one gas-inlet of the container may be connected to two or more gas-inlets of the photobioreactor.

The container may comprise at least one container-outlet. The at least one container-outlet may be in fluid connection with at least one photobioreactor-outlet. Two or more photobioreactor-outlets may be in fluid connection with one container-outlet.

In an embodiment of the present invention the at least one container-outlet may be a liquid-outlet for removing cultivation broth or cultivation medium from the container; or the at least one container-outlet may be a gas-outlet for removing gasses, e.g. exhaust gas, from the container.

The at least one photobioreactor-outlet may be in fluid connection with the at least one container-outlet.

The at least one photobioreactor-outlet may be a liquid-outlet for removing cultivation broth or cultivation medium from the photobioreactor; or the at least one photobioreactor-outlet may be a gas-outlet for removing gasses, e.g. exhaust gas, from the photobioreactor.

In an embodiment of the present invention the distance between the outer surface of the photobioreactor tube and the light source is less than 4 cm, such as less than 3 cm, e.g. less than 2 cm, such as less than 1 cm, e.g. less than 0.1 cm, such as less than 0.05 cm, e.g. less than 0.01 cm, such as 0 cm.

In yet an embodiment of the present invention, the distance between the outer surface of the photobioreactor tube and the light source is in the range of 0.01-5 cm, such as in the range of 0.025-4 cm, e.g. in the range of 0.05-3 cm, such as in the range of 0.75-2 cm, e.g. in the range of 0.8-1.5 cm, such as about 1 cm.

In a further embodiment of the present invention the photobioreactor tube may be fully encircled or partly encircled by the light source.

When the photobioreactor tube may be encircled or partly encircled by the light source the light source is placed closely to the outer surface of the photobioreactor tube and is surrounding fully or partly the photobioreactor tube.

When the at least one light source may be provided as at least two sheaths surrounding each of the photobioreactor tubes, the at least one light source may be placed on the inside of the sheaths and facing the photobioreactor tubes.

When the at least one light source may be provided as at least two sheaths surrounding each of the photobioreactor tubes, the distance between two or more adjacent photobioreactor tubes as mentioned herein may be determined as the distance between outside surfaces of two or more adjacent sheaths (the outside surface of the sheaths may be the surface facing away from the photobioreactor tube).

In an embodiment of the present invention the at least one light source may be fully encircling the photobioreactor tubes by providing at least two sheaths surrounding each of the photobioreactor tubes and placing the at least one light source between the at least two sheaths and the photobioreactor tube and facing the photobioreactor tubes (directing the light into the photobioreactor tube).

In an embodiment of the present invention wherein the at least one light source is provided without a sheaths surrounding each of the photobioreactor tubes.

The distance of 0 cm between the at least one light source and the outer surface of the photobioreactor tube may mean that the at least one light source may be in contact with the outer surface of the photobioreactor.

It may be preferred that the light source has a distance from the outer surface of the photobioreactor. Preferably, the light source may be placed at least 0.001 cm from the outer surface of the photobioreactor, such as at least 0.005 cm, e.g. at least 0.001 cm, such as at least 0.05 cm, e.g. at least 0.1 cm, such as at least 0.5 cm, e.g. at least 1 cm, such as at least 1.5 cm, e.g. at least 2.0 cm.

In an embodiment of the present invention the light source may be provided in a separate protective transparent element. In the event the light source is provided in a separate protective transparent element the distance between the light source and the outer surface of the photobioreactor tube may be determined from the outer surface of the photobioreactor tube and the outer surface of the protective transparent element.

The distance between the outer surface of the photobioreactor tube and the at least one light source may result in a gap between the outer surface of the photobioreactor tube and the at least one light source.

Preferably, the gap between the outer surface of the photobioreactor tube and the at least one light source is less than 5 cm, e.g. less than 4 cm, such as less than 3 cm, e.g. less than 2 cm, such as less than 1 cm, e.g. less than 0.1 cm, such as less than 0.05 cm, e.g. less than 0.01 cm, such as 0 cm.

The distance between the outer surface of the photobioreactor tube and the light source may preferably be in the range of 0.01-5 cm, such as in the range of 0.025-4 cm, e.g. in the range of 0.05-3 cm, such as in the range of 0.75-2 cm, e.g. in the range of 0.8-1.5 cm, such as about 1 cm.

In an embodiment of the present invention the at least one light source comprises a circular light source.

The circular light source may be a light source capable of illuminate in a 360° direction around the longitudinal direction of the at least one light source. In this way it may be possible to provide a homogenous distribution of the light.

In an embodiment of the present invention wherein the at least one light source may be placed inside the photobioreactor tube. In the event the at least one light source may be placed inside the photobioreactor tube the at least one light source may be placed directly inside the photobioreactor tube and in contact with the cultivation broth; or the at least one light source may be placed in a separate compartment, e.g. in a separate protective transparent element, inside the photobioreactor tube, but without direct contact with the cultivation broth.

When the at least one light source may be placed directly inside the photobioreactor tube and in contact with the cultivation broth, the at least one light source may be encapsulated or coated to allow direct contact with the cultivation broth.

In an embodiment of the present invention the at least one light source may be placed inside the photobioreactor tube and the photobioreactor tube may comprise:
a. a light reflecting material;
b. a diameter of the photobioreactor tube 50 mm or above, such as 75 mm or above, e.g. 100 mm or above, such as 150 mm or above, e.g. 200 mm or above, such as 250 mm or above;
c. at least 2 light sources are placed in parallel and adjacent inside the photobioreactor tube, such as at least 3 light sources, e.g. at least 4 light sources, such as at least 5 light sources, e.g. at least 10 light sources, such as at least 25 light sources, e.g. at least 50 light sources, or
d. a combination hereof.

In addition to the features a-d above the photobioreactor tube may further comprise at least one light source placed outside the photobioreactor tube, e.g. at a distance less than 5 cm from an outer surface of the photobioreactor tube.

In an embodiment of the present invention, the light source may emit light at a wavelength in the range of 200-800 nm, such as at a wavelength in the range of 300-750 nm, e.g. at a wavelength in the range of 400-725 nm, such as at a wavelength in the range of 600-700 nm.

Preferably, the at least one light source emits light only at a wavelength in the range of 200-800 nm, such as at a wavelength in the range of 300-750 nm, e.g. at a wavelength in the range of 400-725 nm, such as at a wavelength in the range of 600-700 nm.

In an embodiment of the present invention the container and/or the photobioreactor further comprises at least one cultivation substrate-inlet.

The cultivation substrate-inlet may be suitable for introducing a cultivation substrate into the photoreactor. In particular, the cultivation substrate-inlet may be suitable for continuously introducing the cultivation substrate during continuous cultivation of one or more phototrophic microorganism to facilitate growth of the one or more phototrophic microorganism.

The cultivation substrate according to the present invention may comprise nutrients required by the one or more phototrophic microorganism to facilitate growth.

In an embodiment of the present invention the cultivation substrate does not comprises a carbon source.

In another embodiment of the present invention the cultivation substrate comprises a carbon source, preferably, a solubilized carbon source.

Depending on the cultivation product to be produced, the skilled person would know how compose the cultivation substrate.

In a further embodiment of the present invention, the container and/or the photobioreactor comprises a cultivation broth-outlet.

In yet an embodiment of the present invention the one or more photobioreactor tubes may have the same, or substantially the same, diameter; and/or the one or more photobioreactor tubes may have the same, or substantially the same, length.

The length (in straight direction) of the one or more photobioreactor tubes may be at least 0.1 m, such as at least 0.2 m, e.g. at least 0.3 m, such as at least 0.4 m, e.g. at least 0.5 m, such as at least 0.75 m, e.g. at least 1.0 m, such as at least 2 m, e.g. at least 3 m, such as at least 4 m, e.g. at least 5 m, such as at least 7.5 m, e.g. at least 10 m.

The length (in straight direction) of the one or more photobioreactor tubes may be at most 15 m, such as at most 13 m, e.g. at most 11 m, such as at most 10 m.

The length (in straight direction) of the one or more photobioreactor tubes may be in the range of 0.1-15 m, such as in the range of 4-13 m, e.g. in the range of 6-12 m, such as in the range of 9-11 m, e.g. about 10 m.

The photobioreactor tubes may be combined to provide a single photobioreactor tube comprising multiple photoreactor tubes, and multiple turns of direction when a cultivation broth may be allowed to flow in the photobioreactor tube.

In an embodiment of the present invention two or more photobioreactor tubes may be placed adjacent to each other. In yet an embodiment of the present invention two or more photobioreactor tubes may be placed in parallel to each other.

Preferably, two or more adjacent and/or parallel photobioreactor tubes may be placed with a distance 10 cm or less between an outer surface of one photobioreactor tube to the outer surface of another and adjacent photobioreactor tube; such as 9 cm or less; e.g. 8 cm or less; such as 7 cm or less; e.g. 6 cm or less; such as 5 cm or less; e.g. 4 cm or less; such as 3 cm or less; e.g. 2 cm or less; such as 1 cm or less; e.g. 0.5 cm or less; such as 0.1 cm or less; e.g. the outer surface of one photobioreactor tube is in contact with the outer surface of another and adjacent photobioreactor tube.

When determining the distance between an outer surface of one photobioreactor tube and the outer surface of another and adjacent photobioreactor tube, the distance is determined from the surfaces of the two adjacent photobioreactor tubes closest to each other.

In an embodiment of the present invention the photobioreactor tubes are stacked allowing each photobioreactor tube (except for the photobioreactor tube placed in the periphery of the photobioreactor tube module) to be adjacent to 4 tubes or 6 tubes.

In the present context the term “adjacent” may relate to the neighbouring photobioreactor tubes encountered in perimetric scans with increasing distance around the photobioreactor tube cross section.

Preferably the container according to the present invention relates to a shipping container. Preferably, the shipping container according to the present invention may be designed to move an item (like the photobioreactor, a downstream processing system, the power system, the pump system, the substrates, the cooling system, or the like) from one place (e.g. on the back of a truck or on a ship) to another place (e.g. another truck or ship or to a production facility) without unloading and reloading the item, and preferably without the need unload the item(s) of the container(s) at the production facility.

The container may be constructed of corrugated steel sheets for the side walls. The flooring inside the container may be supported by several steel cross members running horizontally across the width of the container.

The container may be shaped as a rectangular prism. Preferably, the container may be shaped as a rectangular prism and the photobioreactor tubes may be placed in the longitudinal direction of the longest side of the rectangular prism.

In yet an embodiment of the present invention the container is a 20 feet container or a 40 feet container, preferably a 40 feet container.

In a further embodiment of the present invention a flow of cultivation broth in two or more adjacent photobioreactor tubes of the photobioreactor of the present invention may be in opposite directions.

In yet an embodiment of the present invention a flow of cultivation broth in two or more adjacent photobioreactor tubes of the photobioreactor of the present invention may be in the same directions.

The flow of cultivation broth in two or more adjacent photobioreactor tubes may be in opposite directions and the flow of cultivation broth in two or more adjacent photobioreactor tubes are in the same directions.

To provide movement and circulation of the cultivation broth in the photobioreactor a circulation pump may be provided.

In an embodiment of the present invention the pump system, e.g. the circulation pump, may be placed inside the container comprising the photobioreactor or outside the container comprising the photobioreactor.

The circulation pump may be placed outside of the container, whereby a circulation conduit may be included allowing the cultivation broth to be transported outside of the container, through the circulation pump and back into the container again.

Alternatively, the circulation pump may be placed in a separate container (e.g. including similar circulation conduit as described above) and the two containers may be coupled together, allowing for an easy up- or down scale of the production capacity; or the circulation pump may be placed inside the container, together with the photobioreactor. When the circulation pump is placed inside the container, the circulation pump may be placed “inline” on the photobioreactor, or a circulation conduit as described above may be used.

In an embodiment of the present invention a circulation pump may be coupled to the photobioreactor.

In an embodiment of the present invention the container comprises a circulation pump or the container may be coupled to a separate container comprising the circulation pump (a pump container).

It may be desirable to separate the cultivation broth in a liquid phase and a gas phase such as gaseous oxygen, and to isolate a cultivation product from the liquid phase, e.g. providing a biomass which may be further fractionated to obtain e.g. an oil, such as biodiesel, hence a downstream processing container may be provided.

The separation of the cultivation broth in the downstream processing system (placed inside the container comprising the photobioreactor or placed in a separate container—the downstream processing container) may result in a cultivation medium and a cultivation product that may be transported outside of the container, and at least a part of the cultivation medium may be recycled back into the photobioreactor again.

The destination of the cultivation medium obtained from separation of the cultivation broth in the downstream processing system (recircled in the same photobioreactor tube module or introduced into a cultivation medium inlet of another photobioreactor tube module) may be achieved by precipitation, filtration, flotation or gravitational separation and may be manually or automatically controlled.

Preferably, the separation of the cultivation broth may be performed in a downstream processing container. Preferably, the downstream processing container may be coupled to one or more of the containers comprising the photobioreactors which may be in fluid contact via at least one cultivation broth-throughput.

Separation in a separate downstream processing container (e.g. including similar circulation conduit as described above) may allow for an easy up- or down scale of the production capacity; because a single downstream processing container may service several containers comprising the photobioreactor compared to the setup where the downstream processing system may be place inside the container, together with the photobioreactor.

In an embodiment of the present invention a downstream processing system may be coupled to the photobioreactor.

In an embodiment of the present invention the container comprises a downstream processing system or the container may be coupled to a separate container comprising the downstream processing system (a downstream processing container).

In a further embodiment of the present invention the fractionation unit may comprise a first separator capable of separating the cultivation broth into at least one gas phase and a cultivation medium.

The container and/or the photobioreactor may comprise at least one gas-inlet. Preferably, the at least one gas-inlet may include at least one carbon dioxide-inlet (a CO₂-inlet).

The container and/or the photobioreactor may comprise at least one gas-outlet. Preferably, the at least one gas-outlet may include at least one oxygen-outlet (an O₂-outlet).

In a further embodiment of the present invention the first separator may comprise a degassing separator.

The gas phase separated from the cultivation broth may be further separated into individual gasses.

In an embodiment of the present invention the gas phase separated from the cultivation broth may comprise oxygen (O₂) and/or carbon dioxide (CO₂), preferably oxygen (O₂).

In another embodiment of the present invention the container comprises a gas-outlet for liberating the oxygen (O₂) separated from the cultivation broth, preferably separated using the first separator. The liberated oxygen (O₂) may be collected. Preferably, the gas liberated/exhausted from the gas-outlet comprise at least 20% (v/v) O₂; such as at least 25% (v/v) O₂; e.g. at least 50% (v/v) O₂; such as at least 75% (v/v) O₂; e.g. at least 90% (v/v) O₂; such as at least 95% (v/v) O₂; e.g. at least 98% (v/v) O₂.

The carbon dioxide (CO₂) obtained from the first separator may be recircled to the gas-inlet. In an embodiment of the present invention the container may comprise a recirculation conduit transferring carbon dioxide from the first separator to the one or more gas-inlet, such as the one or more CO₂-inlet(s).

The container and/or the photobioreactor may comprise at least one cultivation product-outlet. Preferably, the at least one cultivation product-outlet may include at least one biomass-outlet (a biomass-outlet).

When applying the cultivation broth to the fractionation unit various fractions may be obtained.

The cultivation broth may be provided to a first separator. From the first separator a biomass may be obtained, and a cultivation medium may be obtained.

In an embodiment of the present invention the cultivation medium obtained from the cultivation broth may be recircled to the same photobioreactor or the cultivation medium obtained from one photobioreactor may be introduced into a cultivation medium inlet of another photobioreactor.

In the context of the present invention the term “cultivation broth” relates to the liquid phase circulated in the photobioreactor allowing the microorganism to growth, including water, microbial cells (in the present invention one or more phototrophic microorganism), nutrients, carbon source, etc.

In the context of the present invention the term “cultivation medium” relates to a cultivation broth where at least the biomass has been removed.

In the context of the present invention the term “cultivation substrate” relates to the medium added to the photobioreactor comprising nutrients, minerals, vitamins and which promotes cultivation of a desired microorganism resulting in the desired biomass.

In an embodiment of the present invention the cultivation substrate does not include recycled cultivation medium, or the cultivation substrate comprise the combination new cultivation substrate and recycled cultivation medium.

In an embodiment of the present invention, the container comprises at least one cultivation medium-throughput allowing the cultivation medium or the cultivation broth (if biomass has not been isolated before running through the cultivation medium-throughput) to run from one container into another container.

In an embodiment of the present invention the one or more light source may be placed as at least two sheaths surrounding each of the photobioreactor tubes.

In a further embodiment of the present invention the one or more light source may be provided in the in the space between the photobioreactor tubes.

The light source may be provided in the cavities created between the photobioreactor tubes.

In an embodiment of the present invention the light source may be provided in a polygonic shaped space between the surrounding photobioreactor tubes where the sides are circular shaped or parts hereof. The polygonic shaped space provided by the present invention may three sides or four sides.

In a further embodiment of the present invention polygonic shaped space between the three sides or the four sides provided by the photobioreactor tubes placed adjacent to each other may comprise a circle having a diameter of at most 50 mm, such as at most 25 mm, e.g. at most 15 mm, such as at most 10 mm, e.g. at most 7.5 mm, such as at most 5 mm.

In a further embodiment of the present invention the one or more photobioreactor tubes may have an outside diameter of at least 10 mm, such as at least 20 mm, such as at least 30 mm, e.g. at least 40 mm, such as at least 45 mm, e.g. at least 50 mm, such as at least 60 mm, e.g. at least 70 mm, such as at least 80 mm, e.g. at least 90 mm, such as at least 100 mm, e.g. at least 150 mm, such as at least 200 mm, e.g. at least 250 mm, such as at least 300 mm, e.g. in the range of 20-250 mm, such as in the range of 25-150 mm, e.g. in the range of 30-100 mm, such as in the range of 35-70 mm, e.g. in the range of 40-60 mm.

In yet an embodiment of the present invention the one or more photobioreactor tubes may have an outside diameter of at least 10 mm, such as at least 20 mm, such as at least 30 mm, e.g. at least 40 mm, such as at least 45 mm, e.g. at least 50 mm, such as at least 60 mm, e.g. at least 70 mm, such as at least 80 mm, e.g. at least 90 mm, such as at least 100 mm, e.g. at least 150 mm, such as at least 200 mm, e.g. at least 250 mm, such as at least 300 mm, and an outside diameter of at most 100 mm, such as at most 90 mm, e.g. at most 80 mm, such as at most 70 mm, e.g. at most 60 mm, such as at most 50 mm, e.g. at most 45 mm.

The two or more photobioreactor tube modules may be serially connected, such as 3 or more photobioreactor tube modules, e.g. 4 or more photobioreactor tube modules, 5 or more photobioreactor tube modules, 10 or more photobioreactor tube modules, 15 or more photobioreactor tube modules, 20 or more photobioreactor tube modules, 5 or more photobioreactor tube modules.

A preferred embodiment of the present invention relate to a system comprising one or more containers according to anyone of claims 1-10, wherein the one or more containers are in fluid contact via at least one cultivation medium-throughput and/or via at least one cultivation broth-throughput, with:
one or more further containers according to anyone of claims 1-10;
one or more downstream processing container;
one or more pump containers; and/or
one or more containers comprising the cooling system.

A further preferred embodiment of the present invention relates to a system comprising two or more containers according to the present invention, wherein the two or more containers are in fluid contact via at least one cultivation medium-throughput and/or via at least one cultivation broth-throughput.

In the system of the present invention the two or more containers may include two or more containers comprising:
the photobioreactor; or
one or more containers comprising the photobioreactor in combination with one or more pump-container(s) and/or one or more downstream processing containers and/or one or more containers comprising a cooling system and/or one or more cultivation substrate container.

The system may further comprise a pump system (e.g. including the circulation pumps).

In an embodiment of the present invention the pump system may be provided as an integrated part of the at least one container(s) comprising the photobioreactor.

In another embodiment of the present invention the pump system may be provided in an individual container—in a pump container—in fluid connection with the at least one container comprising the photobioreactor.

The presence of at least one cultivation medium-throughput and/or one or more cultivation broth-throughput in the container allows two or more containers to be stacked (horizontally stacked or vertically stacked). The ability to stack containers makes it easily to up- and down-scaling the process according to individual needs.

This ability to stack the containers is provided by arranging the cultivation medium-throughput from a first container to be aligned with the cultivation medium-throughput of a second container. The flow through the cultivation medium-throughputs or cultivation broth throughputs may be controlled using a valve or a pump. The valves and/or pumps may be mechanical managed or automatically controlled.

A preferred embodiment of the present invention relates to a method for producing at least one cultivation product from culturing one or more phototrophic microorganism, the method comprising the steps of:
(i) Providing the one or more phototrophic microorganism to a photobioreactor of the one or more container according to the present invention or the system according to the present invention;
(ii) Allowing the one or more phototrophic microorganism to ferment under time, temperature and illumination conditions suitable for providing a cultivation broth comprising the cultivation product; and
(iii) Isolating the at least one cultivation product from the cultivation broth.

Preferably, the isolation of the at least one cultivation product from the cultivation broth may be done in a container different from the container comprising the photobioreactor—preferably, in at least one downstream processing container.

The method according to the present invention wherein in the range of 0.1-10% (vol/vol) of the total cultivation broth in the photobioreactor may be harvested, such as in the range of 0.25-7.5% (vol/vol), e.g. in the range of 0.5-5% (vol/vol), such as in the range of 0.75-2.5% (vol/vol), e.g. in the range of 1.0-2% (vol/vol), such as about 1.5% (vol/vol).

Preferably, the harvesting of cultivation broth may be continuous harvesting.

In an embodiment of the present invention, harvesting may begin when a predetermined cell density has been reached.

The one or more phototrophic microorganism may comprise one or more phototrophic algae and/or one or more phototrophic bacteria and/or a mixed culture comprising one or more phototrophic algae and one or more phototrophic bacteria.

A phototrophic microorganism or phototrophs may be microorganisms that use light as their source of energy to produce ATP and carry out various cellular processes and they may anabolically convert carbon dioxide (CO₂) into organic material and cultivation products.

The one or more phototrophic algae may be selected from Chlorella vulgaris, Scenedesmus quadricauda, Scenedesmus obliquus, Nannochloris atomus, Nannochloropsis oceanica, Nannochloropsis oculata, Nannochloropsis gaditana, Chlorococcum littorale, Pseudochlorococcum sp., Haematococcus pluvialis, Dunaliella tertiolecta, Neochloris oleoabundans, Phaeodactylum tricornutum, Thalassiosira weisflogii, Thalassiosira pseudonanna, Skeletonema costatum, Nitzschia closterium, Nitzschia pusilla, Stichococcus bacillaris, Tetraselmis suecica, Pavlova lutheri, Chaetoceros calcitrans, Isochrysis galbana, Rhodomonas baltica, Porphyridium cruentum, Botryococcus braunii, Emiliana huxleyi, Spirulina platensis, Synechococcus sp., Synechocystis sp., Euglena gracilis, Parietochloris incisa, or a combination hereof.

The cultivation product may be biodiesel obtained from the biomass produced according to the present invention and/or oxygen.

The biomass may comprise the one or more phototrophic microorganisms. Preferably, the biomass consists essentially of one or more phototrophic microorganism.

In the context of the present invention, the term “comprising”, which may be synonymous with the terms “including”, “containing” or “characterized by”, relates to an inclusive or open-ended listing of features and does not exclude additional, unrecited features or method steps. The term “comprising” leaves the claim open for the inclusion of unspecified ingredients even in major amounts.

In the context of the present invention, the term “consisting essentially of”, relates to a limitation of the scope of a claim to the specified features or steps, and those features or steps, not mentioned and which do not materially affect the basic and novel characteristic(s) of the claimed invention.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

1. A container comprising at least one gas-inlet, a photobioreactor and at least one light source, wherein the at least one gas-inlet of the container is in fluid connection with at least one gas-inlet of the photobioreactor and wherein the at least one light source is placed in a distance less than 5 cm from an outer surface of a photobioreactor tube and/or the at least one light source is placed inside the photobioreactor tube.

2. The container according to claim 1, wherein the container is a shipping container.

3. The container according to claim 1, wherein the container is a 20 feet container or a 40 feet container, preferably a 40 feet container.

4. The container according to claim 1, wherein the container comprises a cultivation substrate inlet, a cultivation medium inlet, and/or a cultivation broth inlet.

5. The container according to claim 1, wherein the container comprises a cooling water inlet.

6. The container according to claim 1, wherein the container further comprising at least one gas-outlet.

7. The container according to claim 6, wherein the at least one gas-outlet of the container is in fluid connection with at least one gas-outlet of the photobioreactor.

8. The container according to claim 1 wherein the distance between the outer surface of the photobioreactor tube and the light source is in the range of 0.01-5 cm, such as in the range of 0.025-4 cm, e.g. in the range of 0.05-3 cm, such as in the range of 0.75-2 cm, e.g. in the range of 0.8-1.5 cm, such as about 1 cm.

9. The container according to claim 1, wherein the photobioreactor comprises a transparent photobioreactor and/or the photobioreactor tube comprises a transparent photobioreactor tube.

10. The container according to claim 1, wherein the at least one light source is placed outside the photobioreactor tube, e.g. at a distance less than 5 cm from an outer surface of the photobioreactor tube.

11. A system comprising one or more containers according to claim 1, wherein the one or more containers are in fluid contact via at least one cultivation medium-throughput and/or via at least one cultivation broth-throughput, with:
one or more further containers;
one or more downstream processing container;
one or more pump containers; and/or
one or more containers comprising the cooling system.

12. A system comprising two or more containers according to claim 1, wherein the two or more containers are in fluid contact via at least one cultivation medium-throughput and/or via at least one cultivation broth-throughput.

13. The system according to claim 12, wherein the system comprising two or more containers further comprises the two or more containers comprising the photobioreactor in combination with one or more pump-container(s) and/or one or more fractionation-container(s) and/or one or more upstream- or downstream processing containers.

14. A method for producing at least one cultivation product from culturing one or more phototrophic microorganism, the method comprising the steps of:
(i) Providing the one or more phototrophic microorganism to a photobioreactor of the one or more containers according to claim 1 or the system;
(ii) Allowing the one or more phototrophic microorganism to cultivate under time, temperature and illumination conditions suitable for providing a cultivation broth comprising the cultivation product; and
(iii) Isolating the at least one cultivation product from the cultivation broth.

15. The method according to claim 14, wherein the isolation of the at least one cultivation product from the cultivation broth is done in a container different from the container comprising the photobioreactor—in at least one downstream processing container.
